(19) United States
(12) Patent Application Publication
(10) Pub. No.: US 2004/0230194 A1
(43) Pub. Date: Nov. 18, 2004

(54) Device and method for attaching soft tissue to bone

(76) Inventors: Mark G. Urbanski, Sarasolta, FL (US); Alberto Bauer, Marbella/Malaga (ES); Jose Lopez, Bradenton, FL (US)

Correspondence Address: RATNERPRESTIA, P O BOX 980, VALLEY FORGE, PA, 19482-0980, US

(21) Appl. No.: 10/719,520

(22) Filed: Nov. 21, 2003

Related U.S. Application Data
(63) Continuation-in-part of application No. 10/167,586, filed on Jun. 12, 2002.

(51) Int. Cl. A61B 17/56; A61F 2/30; A61B 17/58

(52) U.S. Cl. 606/68; 606/60

(57) ABSTRACT

A device, system, and method for attaching soft tissue to bone is provided. The system includes an anchoring device and delivery device which allow a surgeon to achieve two different objectives during reattachment of tissue to bone. The system allows grasping and manipulation of the tissue to achieve proper location and tension on the tissue, and also attachment of the tissue to the bone after the desired location and tension are achieved. The anchoring device comprises a base, and at least two tissue grabbing members, and a core. Also included is a method of using the system to reattach soft tissue to bone.

This application is a Continuation-In-Part application of U.S. application Ser. No. 10/167,586 (pending), which is incorporated herein by reference.

### FIELD OF THE INVENTION

The present invention relates to medical devices and more specifically to a device and method for attaching soft tissue to bone.

### BACKGROUND OF THE INVENTION

There are several devices and methods known for attaching (or reattaching) soft tissue to bone. These devices and methods have been developed largely in response to the relatively common injuries associated with shoulders and knees whereby soft tissues, including ligaments, are torn or otherwise separated from the bone to which they are attached. Such an injury leads to chronic instability in the joint which often requires surgical intervention.

Surgical intervention conventionally involves the use of arthroscopic devices which use a cannula through which cameras and surgical devices are passed and used at the site of repair. These methods and devices have been designed for low trauma and faster recovery time for the patient.

Through the cannula, in addition to visualization devices such as cameras, various tools have been developed to reattach the torn soft tissue to the bone. Various anchors have been devised for attaching the torn tissue to the bone. One particular technique involves the insertion of an anchor into the bone. The anchor inserted either has sutures attached or means for attaching sutures to the anchor. The sutures are connected to the torn tissue and then tightened to allow contact of the tissue to the bone. The tissue and bone eventually reattach through natural healing process.

Such methods, however, have drawbacks. One such drawback is the fact that a surgeon must often use sutures to attach tissue to bone. Another such drawback is that the “pull-out strength” is often lower than desired. “Pull-out strength” is defined qualitatively as the force necessary to pull the anchor out of the bone to which it has been attached. Yet another drawback relates to “break-away strength.” As noted above, much of the prior art relies on sutures, which introduce another potential weakpoint. “Break-away strength” is defined qualitatively as the force necessary to break the suture. Still yet another drawback of the prior art is that the surgeon must use one device for locating and moving the torn soft tissue to the place of reattachment and a second tool or device for actually attaching the tissue. This is especially deleterious because the degree of stretching, or tautness, of the tissue at the time of reattachment must be precise to achieve proper healing and functionality of the joint after healing. Thus, the surgeon must be able to adjust the amount of tension placed on the ligament just prior to its reattachment. Having to use two different devices during placement, therefore, can lead to longer surgery and generally more room for error in tissue reattachment.

Still other devices allow for both tissue movement and anchoring, but do so only by scraping or puncturing tissue before working it into a predrilled hole in the bone. Such devices are deleterious to the tissue which is stabbed or scraped, and do not allow easy adjustment of tension.

### SUMMARY OF THE INVENTION

The present invention includes devices, systems, and methods for attaching soft tissue to bone. The system allows the surgeon to achieve two different objectives during reattachment of the tissue to the bone. The same system allows grasping and manipulation of the tissue to achieve proper location of, and tension on, the tissue, and also attachment of the tissue to the bone after the desired location and tension are achieved. The system is comprised of an anchoring device and delivery device. The anchoring device, in its simplest embodiment, comprises an anchor and a core. The anchor is comprised of a base and two opposing, inwardly biased, tissue grasping members extending from the base, each tissue grasping member having a relaxed, inwardly biased position, a partially expanded intermediate position, and an expanded, locked position. The tissue grasping members are closably expandable between the relaxed, inwardly biased position and the intermediate position. The core has a central axial opening and is disposed within the anchor and is moveable between a proximal position corresponding to the inwardly biased position of the tissue grasping members and a distal position corresponding to the expanded, locked position of the tissue grasping members.

Also included in the present invention is a method for reattaching tissue to bone comprising the steps of grasping a portion of soft tissue between two opposing tissue grasping members, inserting the tissue grasping members along with the grasped portion of soft tissue into a hole in a bone, and anchoring the device within the hole into which it was inserted by expanding the tissue grasping members. This method preferably includes the step of advancing the core into a distal position which causes the expansion of the tissue grasping members.

### BRIEF DESCRIPTION OF THE DRAWING

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1 is a cross-sectional view of an anchor in accordance with the present invention;

FIG. 2 is a cross-sectional view of an anchor and core in accordance with the present invention;

FIG. 3 is a cross-sectional view of the anchor and core of FIG. 2 but with the core moved distally into a locked position;

FIG. 4 is an orthogonal view of an anchor having two tissue grasping members and four bone engaging members in accordance with the present invention;

FIG. 5A is a cross-sectional view of an anchoring device of the present invention disposed on the end of a delivery device with the anchor partially spread;

FIG. 5B is an orthogonal view of the distal end of an applicator in accordance with a delivery device according to the present invention;

FIG. 6 is a cross-sectional view of an anchoring device of the present invention disposed on the end of a delivery device with the anchor in a relaxed, inwardly biased position;

FIG. 7 is a cross-sectional view of an anchoring device of the present invention disposed on the end of a delivery device with the anchor partially spread to almost its locked position;

FIG. 8 is a cross-sectional view of an anchoring device of the present invention disposed on the end of a delivery device with the anchor in its locked position;

FIG. 9 is a cross-sectional view of soft tissue attached to bone;

FIG. 10 is a cross-sectional view of soft tissue tom from bone;

FIG. 11 is a cross-sectional view of bone being drilled in preparation for use of the present invention;

FIG. 12 is a cross-sectional view of bone being drilled in preparation for use of the present invention;

FIG. 13 shows the first step of gathering tissue in accordance with the present invention where a push rod is extended distally to open the tissue grasping members;

FIG. 14 shows the step of grasping soft tissue with the tissue grasping members by moving the push rod proximally to allow closing of the inwardly biased tissue grasping members;

FIG. 15 shows the step of pulling the grasped soft tissue to the location for which reattachment is desired;

FIG. 16 shows the step of inserting the soft tissue into the bone;

FIG. 17 is a close-up view of part of that which is shown in FIG. 16;

FIG. 18 shows the step of expanding the tissue grasping members into the bone by advancing he push rod distally;

FIG. 19 shows the locking of the anchor in place by advancing the push rod sufficiently distally such that the core locks into place;

FIG. 20 shows the step of removing the delivery device;

FIG. 21 is a cross-sectional view of the device after it has been locked in place;

FIG. 22 shows one embodiment of the distal ends of the tissue grasping members in accordance with the present invention;

FIG. 23 shows an alternative embodiment of the distal ends of the tissue grasping members in accordance with the present invention from that shown in FIG. 22;

FIG. 24 shows the twisting of grasped soft tissue to tighten the tissue in accordance with the present invention;

FIG. 25 is a cross-sectional view of an embodiment of the present invention which uses a plug to insert a second piece of soft tissue into an anchoring device previously inserted; and

FIG. 26 is a cross-sectional view of an embodiment of the present invention which uses a second anchoring device in accordance with the present invention to insert a second piece of soft tissue into an anchoring device previously inserted.

### DETAILED DESCRIPTION OF THE INVENTION

The present invention includes devices, systems, and methods for reattaching soft tissue to bone. Although many places in a human or animal body have tissue to bone connection, the present invention is particularly well suited for repairs to the shoulder or knee joints such as reconstructing the anterior cruciate ligament or repairing a dislocated shoulder or torn rotator cuff.

Generally, the present invention includes an anchoring device which allows grasping and manipulation of the tissue to achieve proper location and tension on the tissue, and also attachment of the tissue to the bone after the desired location and tension are achieved. The anchoring device, in its simplest embodiment, comprises an anchor and a core. The anchor is comprised of a base and two opposing, inwardly biased, tissue grasping members extending from the base, each tissue grasping member having a relaxed, inwardly biased position, a partially expanded intermediate position, and an expanded, locked position. The tissue grasping members are closably expandable between the relaxed, inwardly biased position and the intermediate position. The core has a central axial opening and is disposed within the anchor and is moveable between a proximal position corresponding to the inwardly biased position of the tissue grasping members and a distal position corresponding to the expanded, locked position of the tissue grasping members.

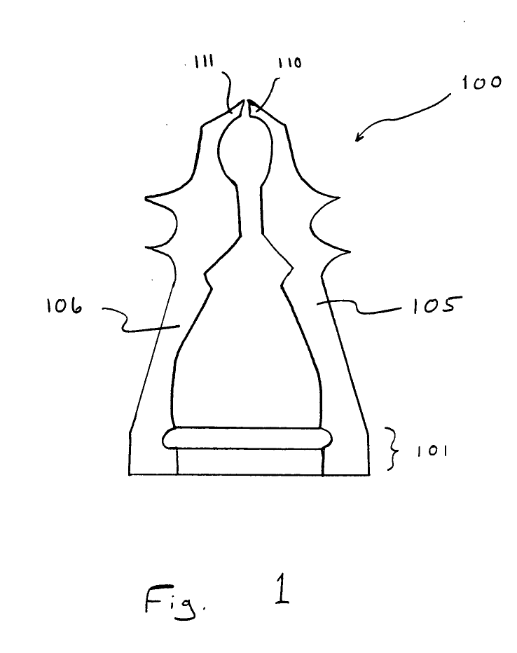

FIGS. 1-3 show an exemplary embodiment of the anchoring device according to the present invention. FIG. 1 shows a cross section of anchor 100 with base 101 supporting two opposing, inwardly biased, tissue grasping members 105 and 106. Each tissue grasping member 105 and 106 in FIG. 1 is in its relaxed, inwardly biased position, with tissue grasping proximal ends 110 and 111 nearly touching. It is not necessary that the distal ends 110 and 111 touch, so long as the space separating them, if any, is small enough to allow the grasping of tissue during use. This aspect will be explained in more detail below.

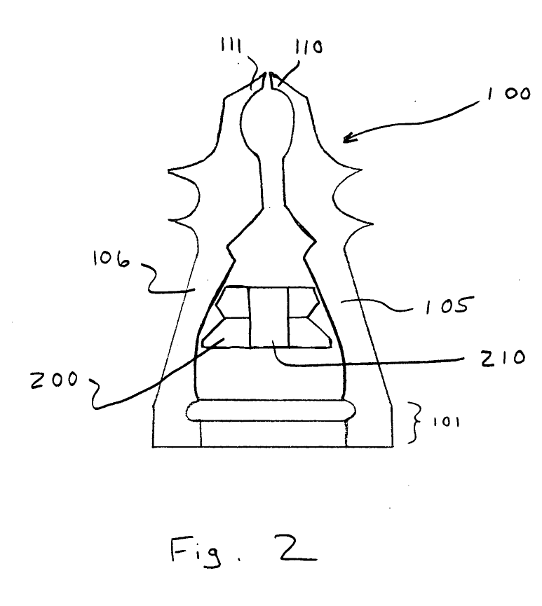

FIG. 2 shows the anchor 100 with core 200 disposed therein. Core 200 has central axial opening 210 disposed through its center. The purpose of this opening will be described in more detail below. As in FIG. 1, FIG. 2 shows grasping members 105 and 106 in their relaxed, inwardly biased position.

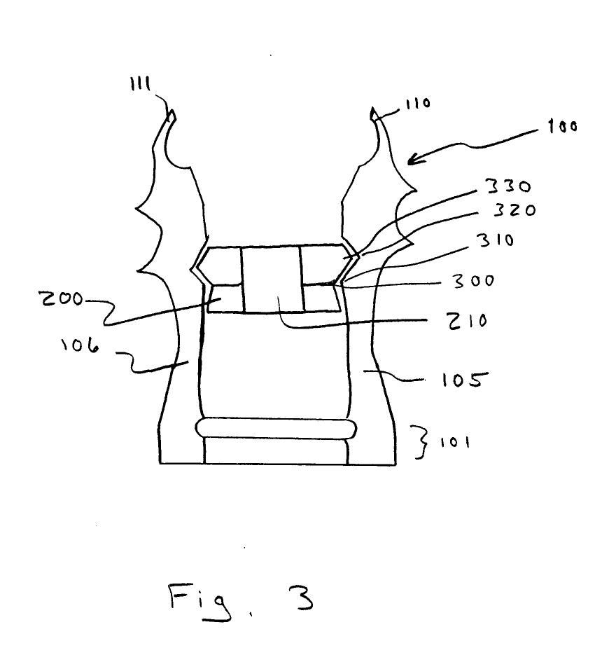

FIG. 3 shows core 200 after it has been moved distally into a distal position where it locks tissue grasping members 105 and 106 in their expanded, locked position. It is seen from FIG. 3 that female groove 300 in core 200 receives male projection 310 from the inner side of tissue grasping members 105 and 106. As added security against dislodgment or over-insertion, a male projection 330 from core 200 can also be used, as is shown in FIG. 3, to mate with female groove 320 on the inner side of tissue grasping members 105 and 106. It should be noted, however, that only one male/female mating is needed to secure core 200 within anchor 100.

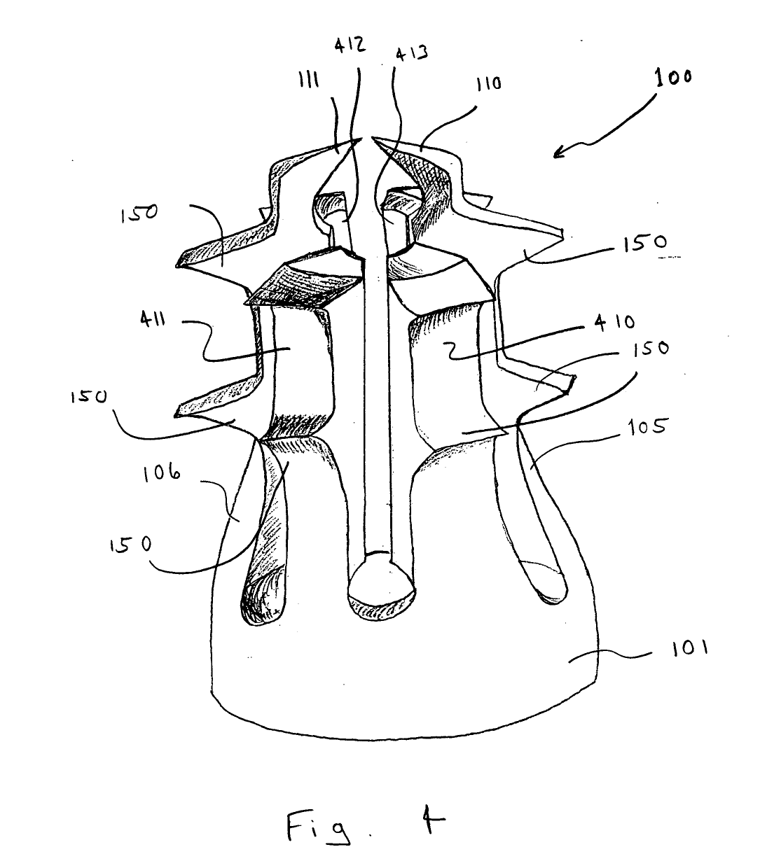

FIG. 4 shows anchor 100 from an angle such that tissue grasping members 105 and 106 are shown diametrically disposed from one another in their relaxed, inwardly biased position. Bone engaging members 410, 411, 412, and 413 are also shown, two each on either side of tissue grasping members 105 and 106. Bone engaging members 410, 411, 412, and 413 do not extend distally from base 101 as far as tissue grasping members 105 and 106, whose tissue grasping proximal ends 110 and 111 extend beyond the distal most part of bone engaging members 410, 411, 412, and 413. The function of these bone engaging members will be addressed in more detail below.

The anchor and core may be made from a number of different materials, so long as the material used for the anchor is pliable enough to allow movement between the unexpanded and expanded positions. Preferably, the anchoring device is made from titanium or other suitable, biocompatible metal or alloy. More preferably, the device is made from nitinol (a nickel-titanium alloy). Alternatively, the device may be made of a biodegradable polymer such as a polylactide based copolymer. Preferred among these biodegradable polymers are poly(l-lactide) (PLLA) and poly(dl-lactide) (PDLLA). More preferred are blends of these polymers, including a 70% PDLLA/30% PLLA blend.

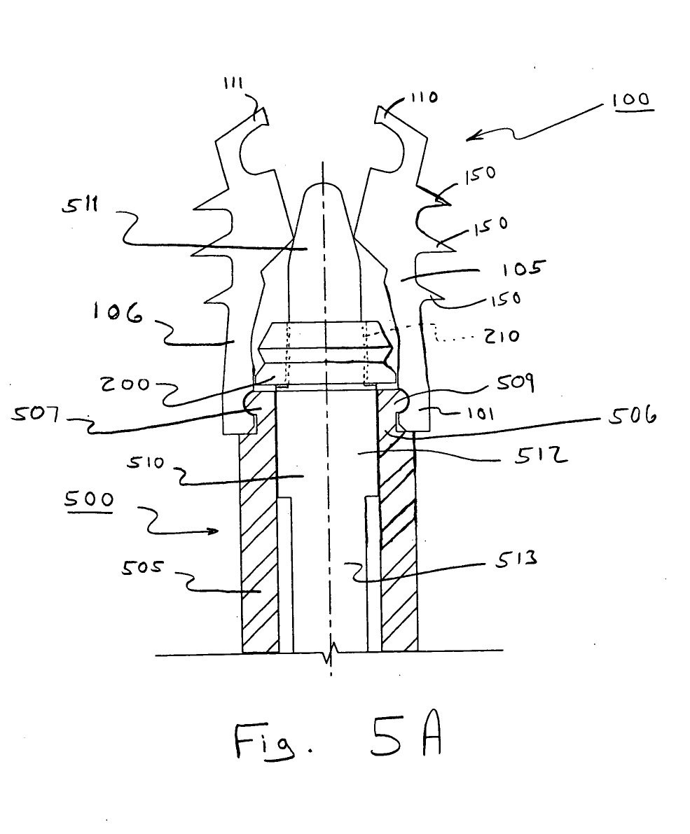

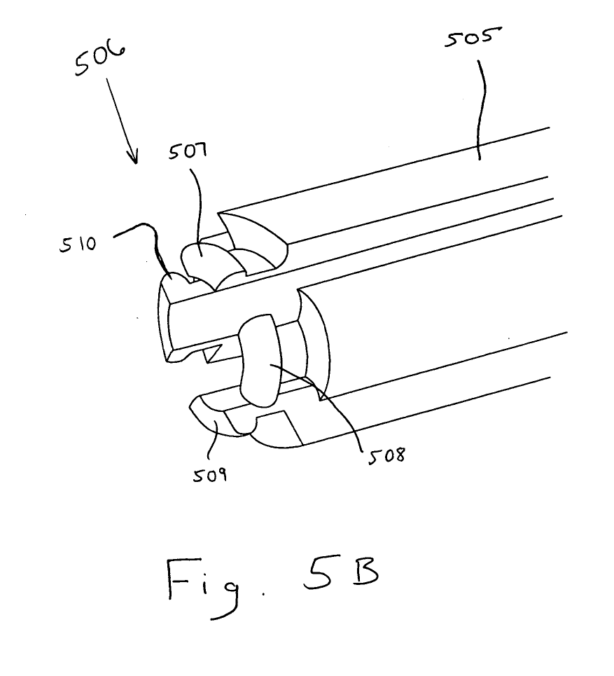

An exemplary embodiment of the anchoring device of the present invention will now be addressed in conjunction with a suitable delivery device. FIG. 5A shows anchor 100 and core 200 removably attached to the end of delivery device 500. This particular embodiment of delivery device 500 is comprised of applicator 505 having a distal end 506, and a push rod 510 slidably and removably disposed coaxially within applicator 505. Push rod 510, in this embodiment, has three regions, namely distal end 511, central region 512, and proximal shaft 513. Central region 512 has a larger diameter than proximal shaft 513. The reason for this change in diameter will be explained in more detail below. Each of the two delivery device elements (applicator 505 and push rod 510) is longitudinally slideable with respect to the other element along a common, central axis, indicated by the dotted line in FIG. 5A.

Distal end 506 of applicator 505 is constructed to be biased inwardly toward the central axis such that push rod central region 512 applies an outward force with respect to applicator distal end 506 so long as push rod central region 512 is disposed as shown in FIG. 5A. It can be seen in FIG. 5A that applicator distal end 506 comprises male projections 507 and 509 around its circumference such that a female groove in base 101 of anchor 100 receives the male projection when applicator distal end 506 is forced outward against its bias by push rod central region 512.

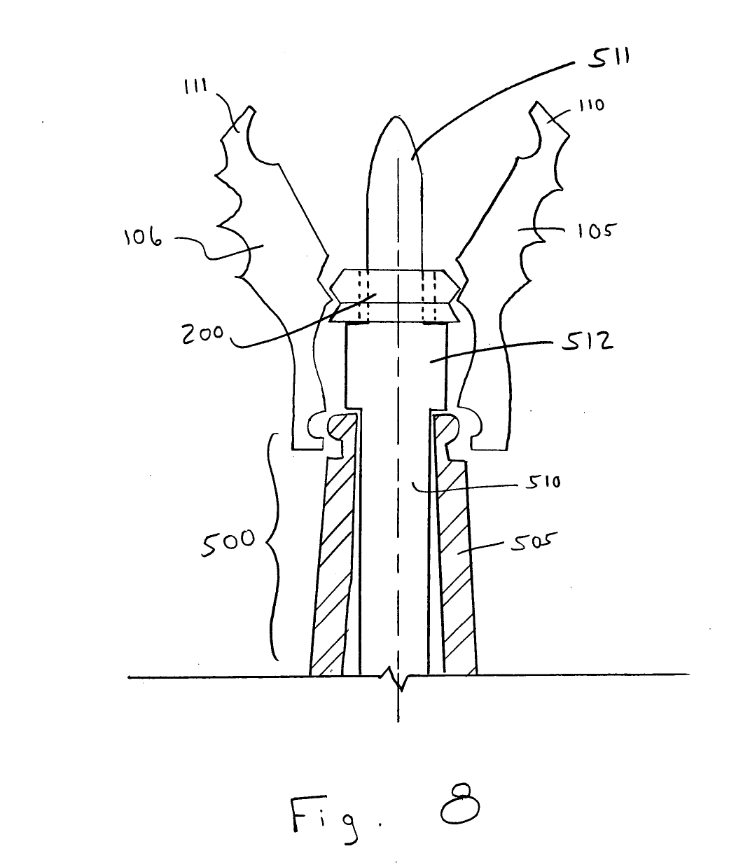

FIG. 5B shows a view of distal end 506 of applicator 505. It is seen that, in this embodiment, four male projections 507, 508, 509, and 510 comprise distal end 506. This configuration allows the radial movement of male projections. Such movement will allow the release of anchor device 100, as shown for example in FIG. 8 and as discussed in more detail below.

FIG. 5A also shows push rod distal end 511 partially expanding tissue grasping members 105 and 106 from their relaxed, inwardly biased position, to a partially expanded intermediate position. It can be seen from FIG. 5A that as push rod 505 is moved slightly proximally or slightly distally from the position shown, tissue grasping members 105 and 106 will move together or farther apart, respectively. This is due to the tapering of push rod distal end 511. Note also that during such movement at this stage, core 200 stays essentially stationary, as its central axial opening 210 allows for movement of push rod 505.

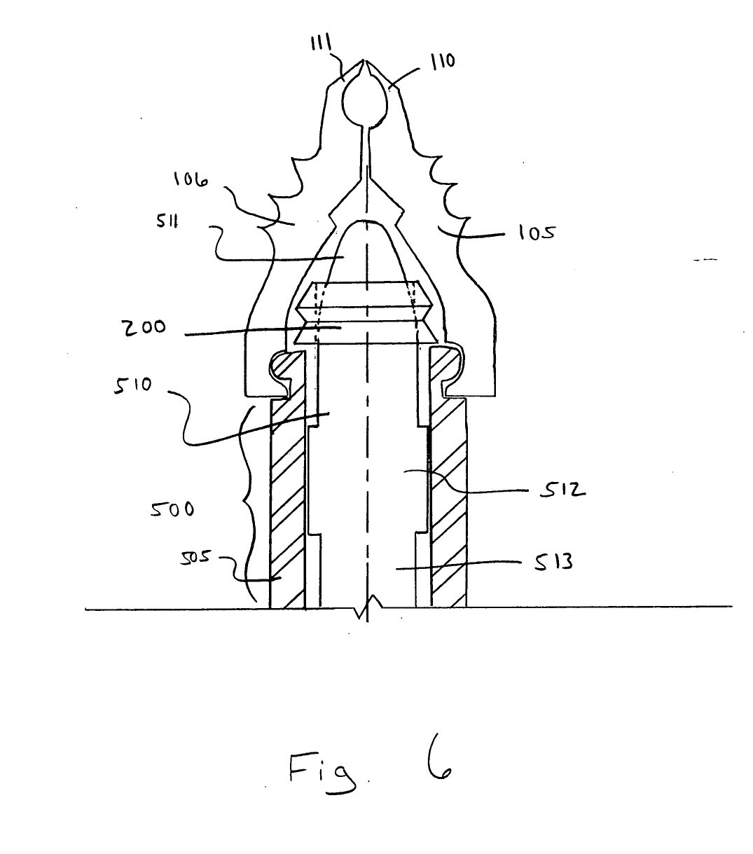

FIG. 6 shows the situation where push rod 505 is moved proximally from the position shown in FIG. 5A such that expanding tissue grasping members 105 and 106 have closed from their intermediate position to their relaxed, inwardly biased position. Note here that push rod central region 512 is still forcing applicator distal end 506 outward, such that the male/female mating as described above is still effectuated to hold anchor 100 in place on the distal end 506 of applicator 505.

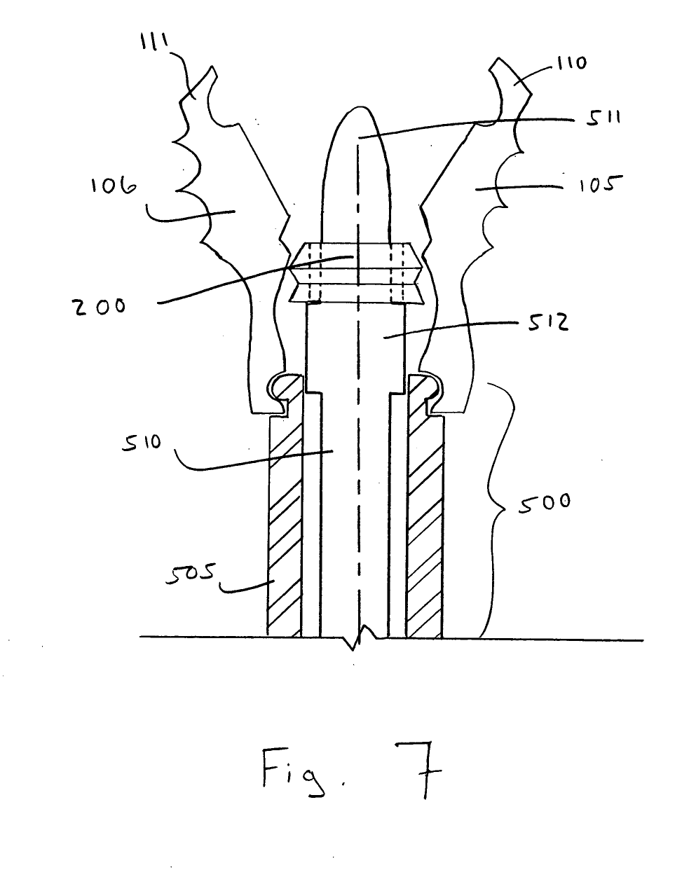

FIG. 7 shows what happens when push rod 510 is moved distally beyond the position shown in FIG. 5A such that the distal end of push rod central region 512 contacts the proximal end of core 200. At this point, further proximal movement of push rod 510 causes core 200 to move distally toward what will be its locking position. FIG. 8 shows core 200 in its locking position, with expanding tissue grasping members 105 and 106 in their fully expanded, locked position. Note here that corresponding grooves and projections on core 200 and the inner surface of tissue grasping members 105 and 106, respectively, act to keep core 200 in the locked position.

FIG. 8 also shows what happens when the proximal end of push rod central region 512 moves past the distal end 506 of applicator 505. Specifically, applicator distal end 506 is allowed to move inwardly under its bias such that the male projections of applicator distal end 506 move out of the female groove of base 101 of anchor 100, freeing anchor 100 from the applicator. At this point, applicator 505 and push rod 510 can be removed.

Moreover, push rod 510 is moveable between three general positions, namely: (1) a proximal position corresponding to the closed, or relaxed, inwardly biased position of tissue grasping members 105 and 106 (FIG. 6); (2) an intermediate position corresponding to an intermediate position of tissue grasping members 105 and 106 (e.g. FIGS. 5A and 7); and (3) the expanded, locked position of tissue grasping members 105 and 106 (FIG. 8). Core 200 may or may not move during intermediate positions, as in, for example, FIGS. 5A and 7, but will move into the locked position once push rod 510 is moved distally far enough to push core 200 into the final, locked position, as shown in FIG. 8.

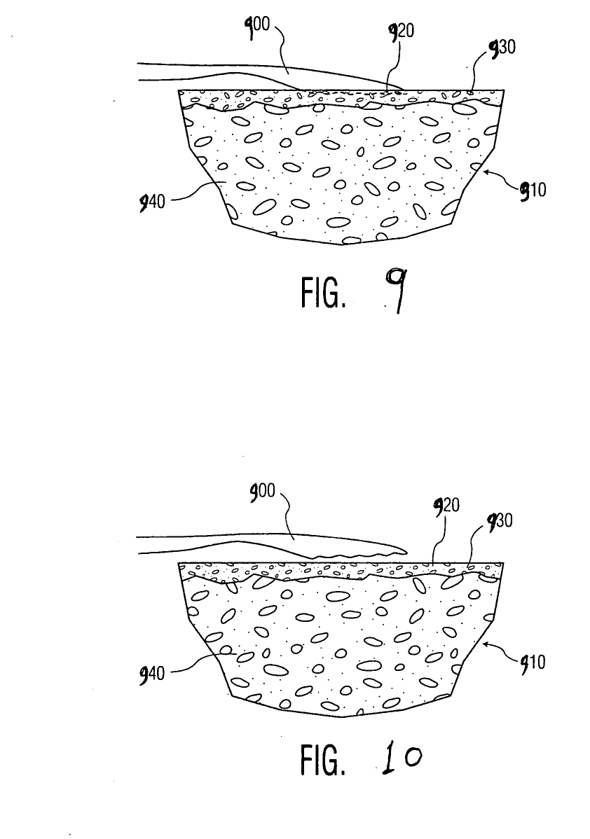

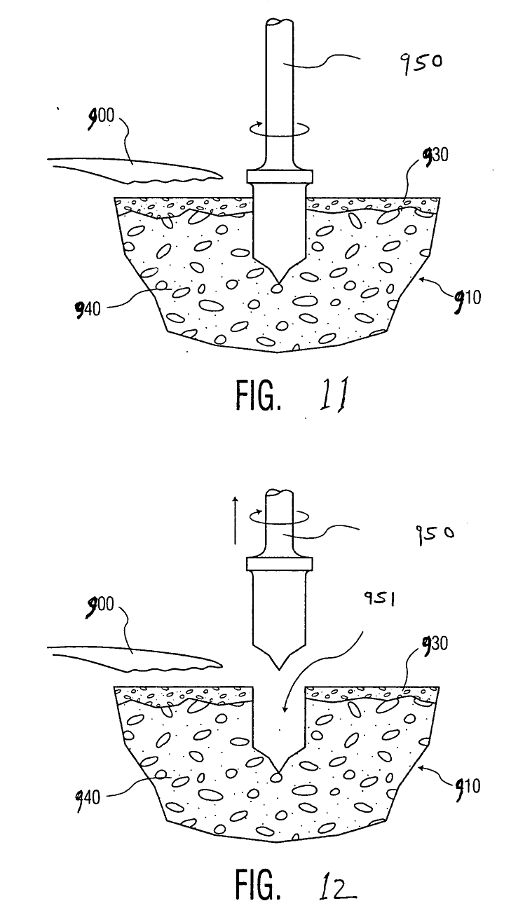

FIGS. 9 to 21 show a method according to the present invention. FIG. 9 shows a piece of normal soft tissue 900 attached to bone 910. Tissue ingrowth area 920 is shown where soft tissue 900 contacts a layer of cortical bone 930. Cancellous bone 940 (softer than the cortical bone) is shown in part below cortical bone 930. FIG. 10 shows soft tissue 900 tom from cortical bone 930.

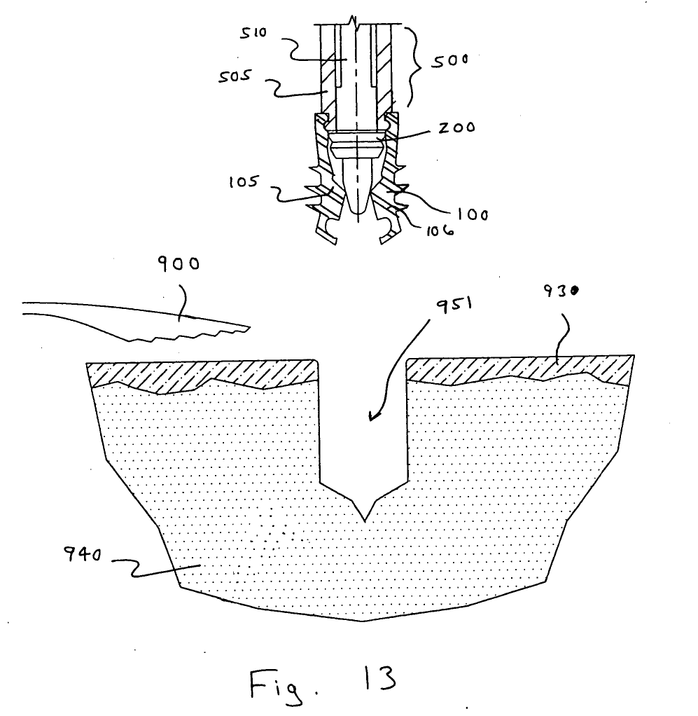

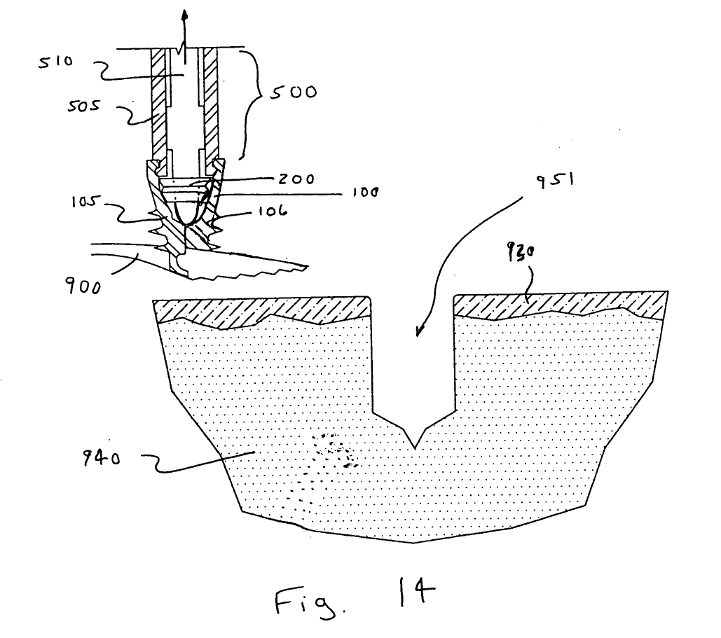

The first step in repairing the tissue after access to the site is achieved by the surgeon is to clean and prepare the bone surface area for drilling. FIG. 11 illustrates drill 950 penetrating cortical bone 930 and cancellous bone 940 to form hole 951, shown in FIG. 12. The types of drill bits and methods for accessing the affected area with drill 950 are well known by those skilled in the art. Important in this step is to insure that hole 951 is drilled to the proper depth. As will be seen more clearly below, anchor 100 must penetrate bone 910 to a depth sufficient to allow effective expansion of anchor 100 along with soft tissue 900 which is forced into hole 951.

FIG. 13 shows the next step, namely gathering soft tissue 900 with the anchoring device comprised of anchor 100 and core 200, which is disposed on the distal end of delivery device 500. The surgeon locates soft tissue 900 for which repair is desired, then opens tissue grasping members 105 and 106 from their relaxed, closed position to an intermediate, tissue grasping position, as shown in FIG. 13. Note that at this point, core 200 has not moved.

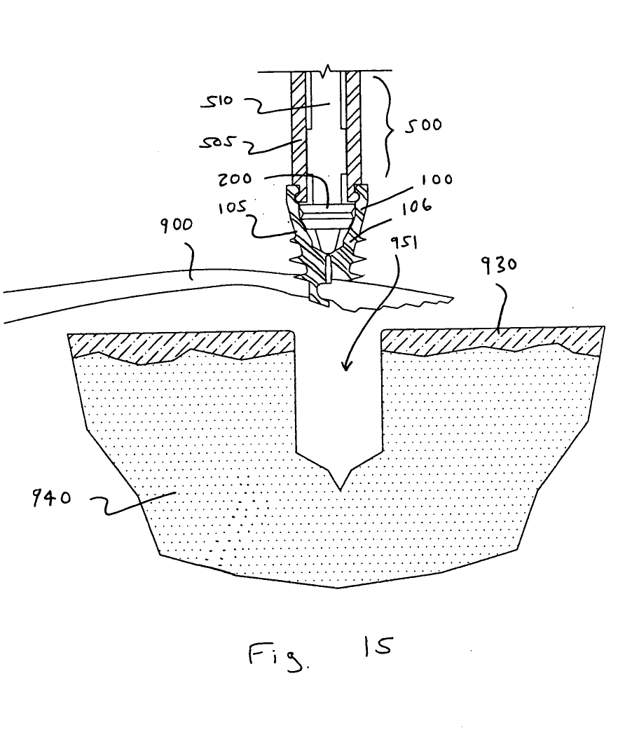

Next, the surgeon grasps soft tissue 900 by moving push rod 510 proximally which allows tissue grasping members 105 and 106 to close under the force of their inward bias around soft tissue 900, as shown in FIG. 14. This allows the surgeon to move and otherwise manipulate the tissue in preparation for its insertion into hole 951.

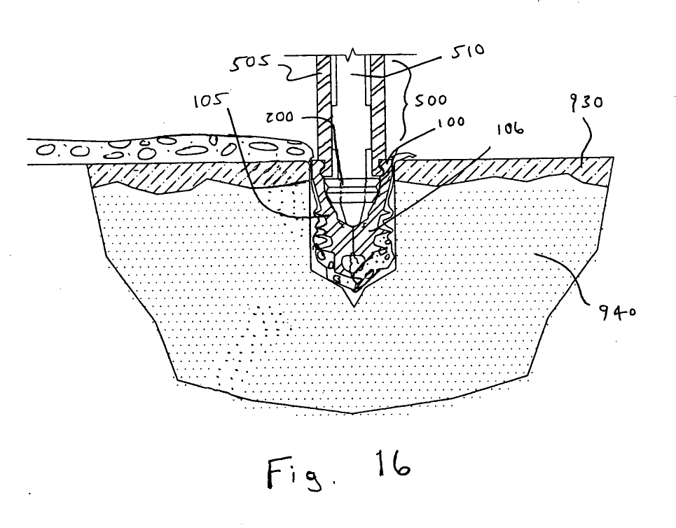

The grasped soft tissue 900, anchor device 100, core 200, and delivery device 500 are then manipulated by the surgeon to position soft tissue 900 above hole 951 as shown in FIG. 15. When soft tissue 900 is pulled over hole 951, the soft tissue undergoes a force which tightens it, and may even stretch it. The surgeon can control the degree of taughtness in a variety of ways. Some of these ways are discussed in more detail below.

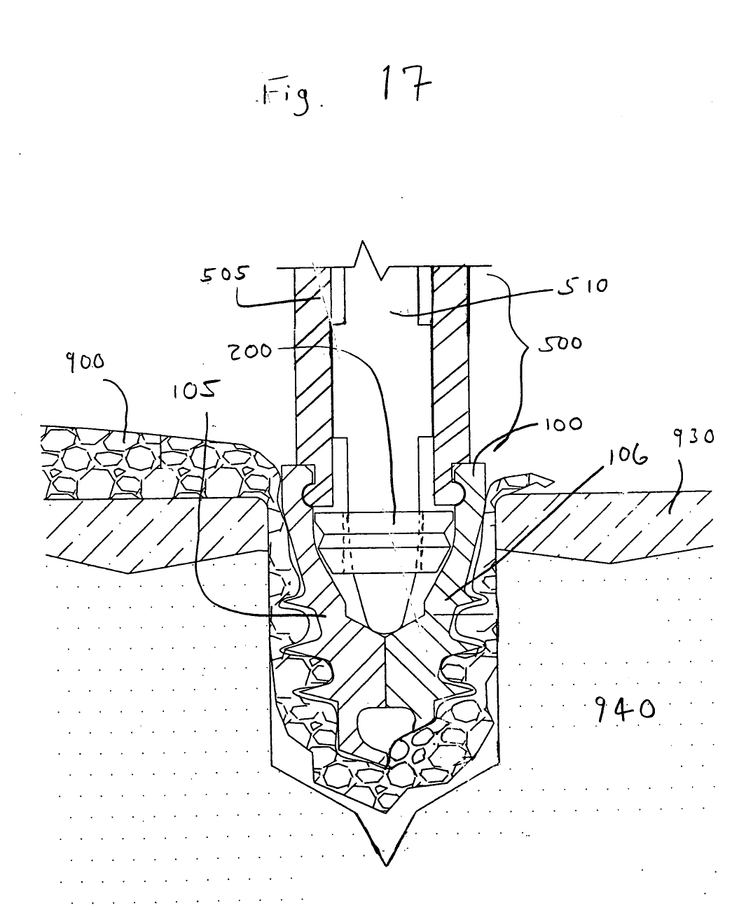

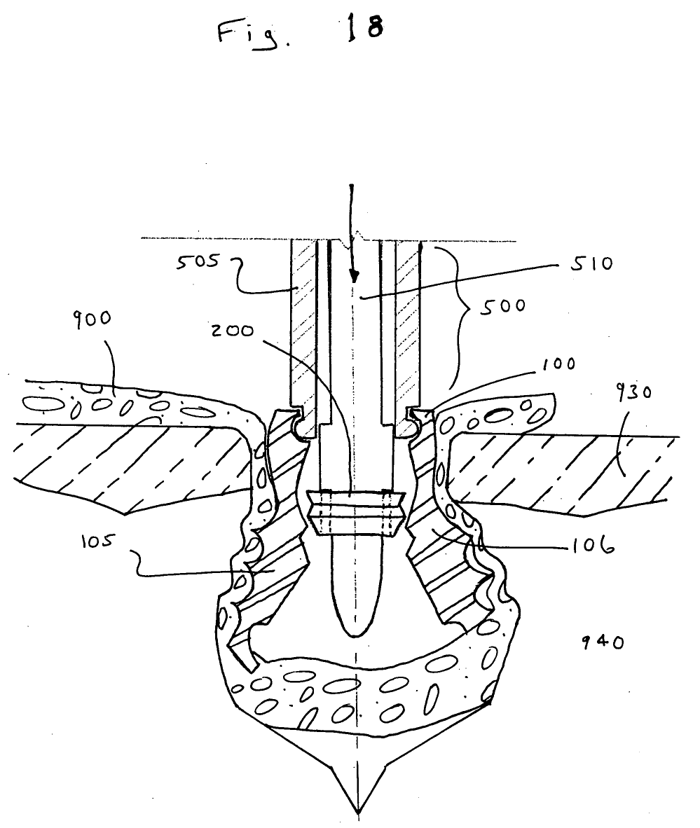

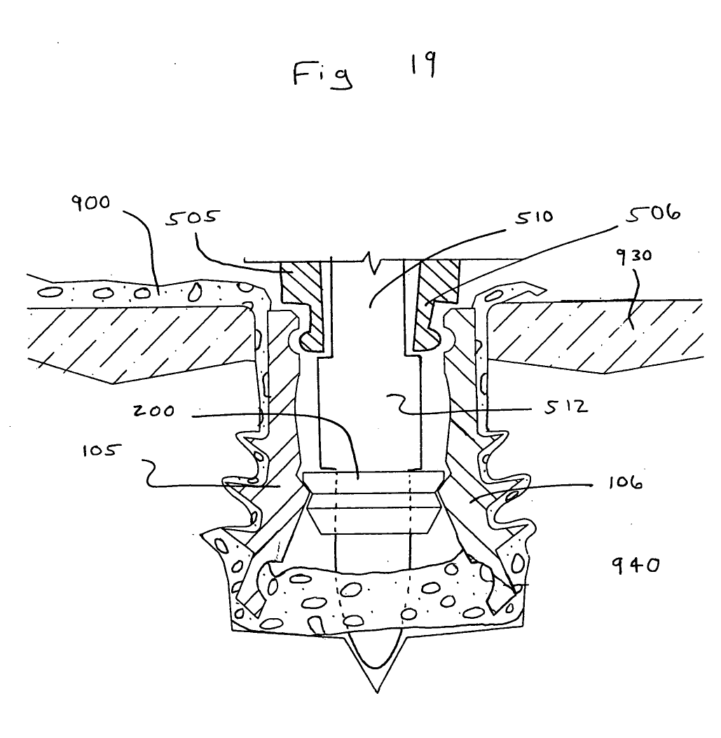

Once the surgeon decides to anchor a piece of soft tissue 900, the surgeon can push the system down into hole 951, as shown in FIG. 16. FIG. 17 is an expanded view of part of FIG. 16. FIG. 18 shows the advancement of push rod 510 during the initial stage of anchoring. At this point, push rod 510 is moved distally which begins the movement of tissue grasping members outward. First, the distal end of push rod 510 causes the expansion of tissue grasping members 105 and 106, but once push rod 510 is moved sufficiently distally, core rod 200 forces tissue grasping members 105 and 106 apart. Such is the case in FIG. 18, which shows sufficient distal advancement of push rod 510 such that core 200 has also begun expansion of tissue grasping members 105 and 106 as it moves toward its locking position. It is noted here that because of the softness of cancellous bone 940, anchor 100 is able to open against the cancellous bone 940, thereby expanding soft tissue 900 beyond the walls of hole 951.

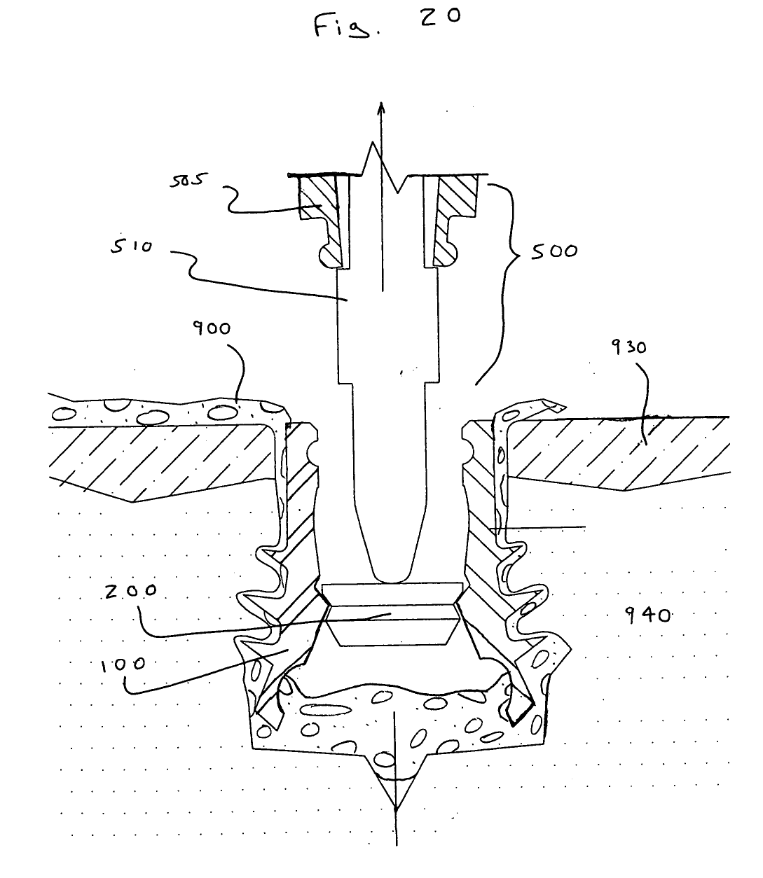

FIG. 19 shows the result of the surgeon advancing push rod 510 distally to a point sufficient to push core 200 into its locking position. FIG. 19 shows core 200 with its female groove having received the male protrusion of the inner surface of tissue grasping members 105 and 106. It can also be seen that male protrusions 507 and 509 at the distal end 506 of applicator 505 are now in their inward position because central region 512 of push rod 510 has moved past the distal end 506 of applicator 505.

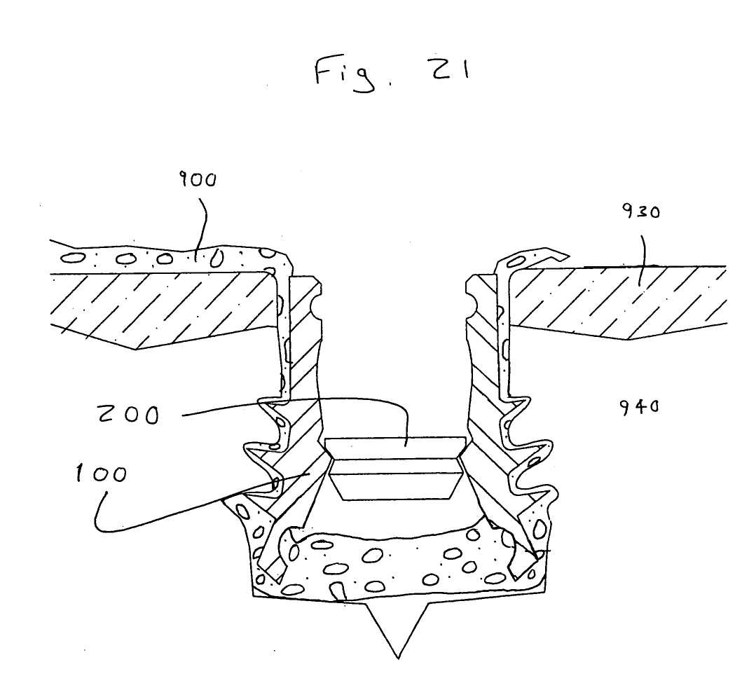

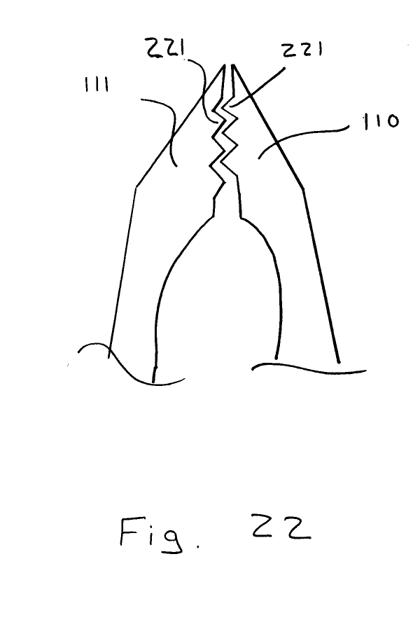

FIG. 20 illustrates what happens when applicator 500 is withdrawn proximally from the site of repair. Anchor 100 and core 200 remain in place, holding soft tissue 900 within cancellous bone 940 and cortical bone 930. FIG. 21 shows what is left in the patient's body. After a period of time for healing has passed, soft tissue 900 will have rejoined cortical bone 930 through normal tissue ingrowth.

Aiding in the anchoring of anchor 100 within the bone are barbs 150 as shown in the embodiment of FIG. 4. These barbs are also preferably present on bone engaging members 410, 411, 412, and 413. The embodiment of FIG. 5A shows three such barbs 150 on each tissue grasping member 105 and 106. Although not shown in the cross section of FIG. 5A, such barbs would also preferably be present on the bone engaging members. The number of barbs is preferably between 1 and 5, although additional barbs could be used.

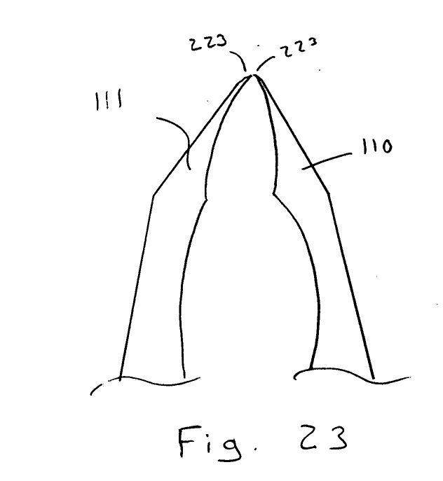

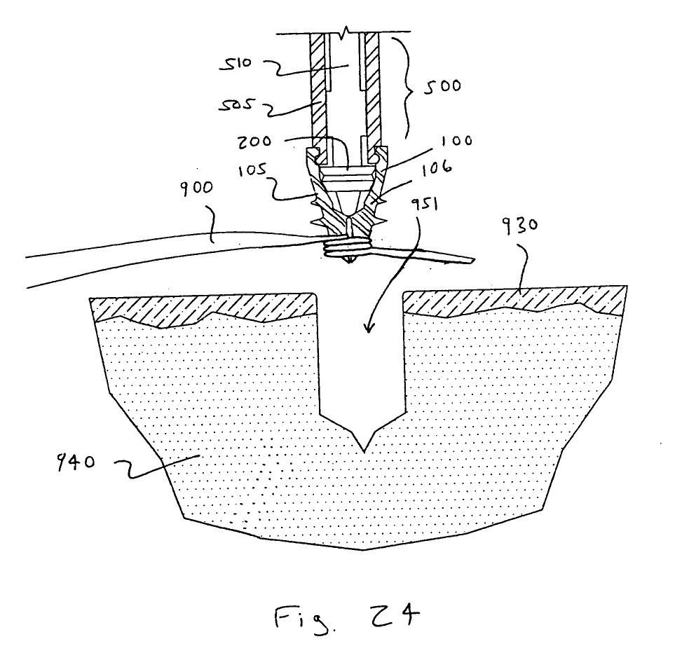

Also included in one embodiment are teeth on the inside surface of the distal tips of tissue grasping members. FIG. 22 shows a close-up of distal tips 110 and 111 having teeth 221. Other configurations can be envisioned which are consistent with the present invention. FIG. 23 shows a set of tips which do not have teeth, but which rely on pointed distal ends 223 for grasping tissue.

As noted above, it is preferred that the surgeon be able to adjust the “taughtness” of the soft tissue, particularly in the case of ligament reattachment, prior to anchoring the tissue into the bone. This can be achieved in a number of ways, some of which are discussed below.

The surgeon can, after initially grasping a piece of soft tissue, twist the entire device, or rotate it, around its central axis, in order to tighten the tissue prior to inserting it into the prepared hole in the bone. This is illustrated in FIG. 24.

Alternatively, the surgeon can grasp, move, partially insert soft tissue into the hole, and then release the tissue and move the device back to regrasp additional tissue and reinsert that tissue over top of the originally inserted tissue. This can be continued until the desired tension in the soft tissue remaining outside of the hole is achieved.

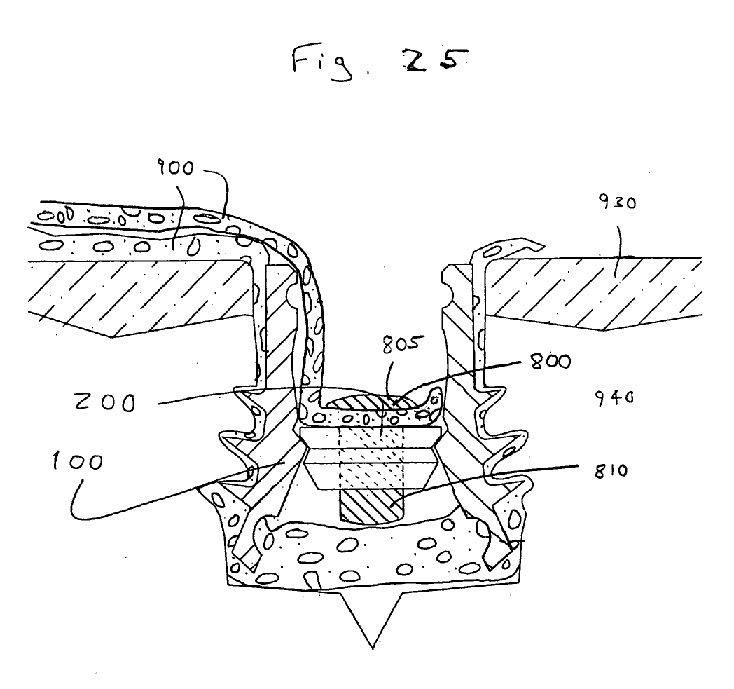

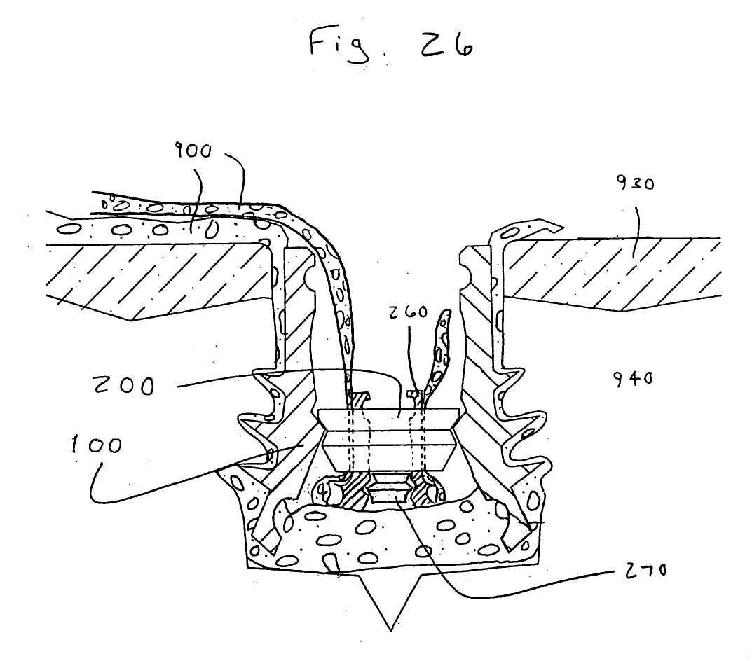

Yet another way to progressively increase tension involves a system similar to that described above, but involves a second anchoring device. In this embodiment, plug 800 having head 805 and shaft 810 could be forced into the first-placed anchoring device as shown in FIG. 25. In this embodiment, plug 800 is simply friction fit into central axial opening 210 of core 200 and is held in place by being compressed within soft tissue 900. Plug 800 may be made of any suitable material, including titanium or biodegradable materials as discussed above. This embodiment requires an additional tool to pull soft tissue over the top opening of core 200 prior to plug 800 being inserted. This method would also require an additional tool for pushing plug 800 into place. Methods and tools for use in placing such a plug are known to those skilled in the art.

Still another way to achieve the desired tension is to anchor a piece of soft tissue into a hole as described above, remove the delivery device and return with a second anchoring device to repeat the process while tightening or gathering more tissue the second and subsequent times. In such a case, each time the tissue is inserted, it could be inserted into a different hole. Alternatively, because core 200 is open in its center, progressively smaller anchoring devices could be used and each inserted into the last-placed anchoring device. Such a system is illustrated in FIG. 26. FIG. 26 shows a second anchor 260 and second core 270 disposed within the anchoring system.

The present invention has been set forth with regard to several preferred embodiments, but the full scope of the invention should be ascertained by the claims that follow.

What is claimed:

1. An anchoring device for attaching soft tissue to bone comprising:
an anchor comprised of:
a base; and
two opposing, inwardly biased, tissue grasping members extending from said base, each said tissue grasping member having a relaxed, inwardly biased position, a partially expanded intermediate position, and an expanded, locked position, wherein said tissue grasping members are closably expandable between said relaxed, inwardly biased position and said intermediate position; and
a core having a central axial opening, said core disposed within said anchor and moveable between a proximal position corresponding to said inwardly biased position of said tissue grasping members and a distal position corresponding to said expanded, locked position of said tissue grasping members.

2. The device of claim 1 wherein said tissue grasping members have an inward facing surface for grasping tissue, and an outer surface having bone engaging barbs.

3. The device of claim 1 further comprising at least two bone engaging members extending from said base, said bone engaging members having a relaxed,
inward position and an expanded, locking position, wherein said distal position of said core corresponds to said locking position of said bone engaging members.

4. The device of claim 3 wherein said bone engaging members have an outer surface with bone engaging barbs extending therefrom.

5. The device of claim 1 wherein said core has a periphery with a female section to engage said inward facing surface of said tissue grasping members and thereby lock said anchoring device in said expanded, locking position.

6. The device of claim 1 wherein said core has a periphery with a male section to engage said inward facing surface of said tissue grasping members and thereby lock said anchoring device in said expanded, locking position.

7. The device of claim 1 wherein the anchor is comprised of a material selected from the group consisting of: titanium, stainless steel, and nitinol.

8. The device of claim 1 wherein the anchor is comprised of a biodegradable polymer.

9. A system for attaching soft tissue to bone comprising:
(a) an anchoring device comprised of:
an anchor having:
a base, and
at least two opposing, inwardly biased, tissue grasping members extending from said base, each said tissue grasping member having a relaxed, inwardly biased position, a partially expanded intermediate position, and an expanded, locking position, wherein said tissue grasping members are closably expandable from said relaxed, inwardly biased position to said intermediate position; and
a core having a central axial opening, said core disposed within said anchor and moveable between a proximal position corresponding to said inwardly biased position of said tissue grasping members and a distal position corresponding to said expanded, locked position of said tissue grasping members; and
(b) a delivery device comprising means for closably expanding said tissue grasping members between said relaxed, inwardly biased position and said intermediate position, and for moving said core from said proximal position to said distal position.

10. The system of claim 9, wherein said delivery device comprises an applicator and an expansion rod movably disposed within the applicator, the expansion rod moveable between first, second, and third positions,
said first position corresponding to said relaxed, inwardly biased position of said tissue grasping members,
said second position corresponding to said partially expanded intermediate position of said tissue grasping members, and
said third position corresponding to said expanded, locking position.

11. The system of claim 9, wherein said delivery device comprises an applicator having a distal end and an expansion rod movably disposed within the applicator;
wherein the distal end of the applicator is radially moveable between a first position for holding the anchoring device and a second position for releasing the anchoring device; and
wherein the expansion rod is moveable between a retracted position which corresponds to the first position of the applicator, and a forward position which corresponds to the second position of the applicator.

12. A method for reattaching tissue to bone comprising the steps of:
grasping a portion of soft tissue between two opposing tissue grasping members;
inserting the tissue grasping members along with the grasped portion of soft tissue into a hole in a bone; and
anchoring the device within the hole into which it was inserted by expanding the tissue grasping members.

13. The method of claim 12 wherein said anchoring step comprises expanding said tissue grasping members by pushing a core distally between said grasping members to wedge said core between said grasping members.

14. The method of claim 13 wherein said core has a central axial opening.

15. The method of claim 14 further comprising the step of inserting a second portion of soft tissue into said central axial opening of said core after said device is anchored in said bone hole.

16. A method for reattaching tissue to bone comprising the steps of:
applying a separation force against two opposing tissue grasping members which are inwardly biased to separate the tissue grasping members;
moving said tissue grasping members around a portion of soft tissue;
removing the separation force to allow said tissue grasping members to close around the portion of soft tissue and thereby grasp the soft tissue;
inserting the tissue grasping members along with the grasped portion of soft tissue into a hole in a bone; and
anchoring the device within the hole into which it was inserted.

17. The method of claim 16 wherein said anchoring step comprises expanding the tissue grasping members radially outward.
